(19) United States
(12) Patent Application Publication
(10) Pub. No.: US 2025/0111908 A1
(43) Pub. Date: Apr. 3, 2025

(54) DIGITAL NATIVE TRIALS MANAGEMENT SYSTEM

(71) Applicant: Intellectual Frontiers LLC, Silver Spring, MD (US)

(72) Inventor: Shahid N. Shah, Silver Spring, MD (US)

(73) Assignee: Intellectual Frontiers LLC, Silver Spring, MD (US)

(21) Appl. No.: 18/977,822

(22) Filed: Dec. 11, 2024

(51) Int. Cl. G16H 10/20; G06Q 10/0631; G16H 40/67

(52) U.S. Cl. CPC G16H 10/20; G06Q 10/06315; G16H 40/67

(57) ABSTRACT

A system for autonomously managing an experimental study using real-world data. The system includes a processor that is configured to generate or receive an adaptive trial protocol defining participant criteria, one or more interventions, one or more outcomes, and respective durations. The processor is configured to facilitate recruitment of one or more participants via one or more digital channels based on assessment of eligibility against the participant criteria. The processor is configured to continuously analyze the real-world data associated with the one or more participants for one or more of adaptive trial adjustments, anomaly detection, and outcome generation.

### BACKGROUND

### Technical Field

The embodiments herein generally relate to systems and methods for conducting experimental studies and, more particularly, to adaptive computer-controlled systems and methods for conducting and managing experimental studies involving participant recruitment.

### Description of the Related Art

Existing methods for conducting experimental studies, including clinical studies, often involve manual recruitment processes, fragmented data collection, and static trial designs mostly requiring manual processes. These cannot dynamically adapt to interim results and varying type of data patterns and require sophisticated processes and expertise to conduct them. Hence, such trials are often conducted in specific authorized sites and under highly specific, rigid formats. These limitations are further compounded by the difficulty in integrating diverse data sources into a cohesive system for analysis and decision-making in real-time.

Additionally, traditional systems lack the capability to autonomously match participants to ongoing studies based on real-time data, thereby increasing study timelines and operational inefficiencies. There is also limited support for facilitating recruitment models that leverage advanced technologies to improve participant involvement with minimal or no manual involvement for managing or conducting the studies.

### SUMMARY

An embodiment herein provides an adaptive software-as-a-service (SaaS)-based system for autonomously managing an experimental study using real-world data. The system includes a processor that is configured to generate or receive a trial protocol defining participant criteria, one or more interventions, one or more outcomes, and respective durations. The processor is configured to facilitate recruitment of one or more participants via one or more digital channels based on assessment of eligibility against the participant criteria. The processor is configured to continuously analyze the real-world data associated with the one or more participants for one or more of adaptive trial adjustments, anomaly detection, and outcome generation.

An embodiment herein provides an adaptive software-as-a-service (SaaS)-based digital system for conducting an autonomous experimental study using real-world data. The system includes a processor configured to execute machine-readable instructions stored on a non-transitory computer-readable medium, wherein the processor is further configured to one of either receive, from a user interface, a trial protocol specifying one or more of participant recruitment criteria, intervention types, outcome measures, and trial duration, or autonomously generate the trial protocol by applying one or more machine learning algorithms to analyze historical study data and multi-dimensional input criteria. The processor is configured to facilitate recruitment of one or more participants by integrating with one or more digital channels, including one or more of a mobile application, a wearable device, and a social networking server. The processor is configured to dynamically assess participant eligibility by processing at least one of real-time physiological measurements and participant-reported inputs. The processor is configured to automatically enroll the one or more participants into the experimental study when their data satisfies the participant recruitment criteria specified in the trial protocol. The processor is configured to execute computational models for analyzing the real-world data during the experimental study to generate one or more trial outcomes.

An embodiment herein provides a method for autonomously managing an experimental study using real-world data through an adaptive software-as-a-service (SaaS)-based system. The method includes generating or receiving a trial protocol, wherein the trial protocol specifies participant criteria, one or more interventions, an outcome, and a duration of the experimental study. The method includes assessing eligibility of the one or more participants against the participant criteria specified in the trial protocol. The method includes facilitating recruitment of the one or more participants through one or more digital channels. The method includes continuously analyzing the real-world data associated with the one or more participants during the experimental study to perform one or more of adaptive trial adjustments, detecting one or more anomalies, and recording one or more outcomes.

### BRIEF DESCRIPTION OF THE DRAWINGS

The features of the disclosed embodiments may become apparent from the following detailed description taken in conjunction with the accompanying drawings showing illustrative embodiments herein, in which:

FIG. 1 illustrates an ecosystem including a clinical trial management system communicatively connected to a plurality of data sources, in accordance with an embodiment.

FIG. 2 illustrates various subsystems and modules implemented within a processor of the clinical trial management system, in accordance with an embodiment.

FIG. 3 illustrates various subsystems of a participation management engine of the clinical trial management system, in accordance with an embodiment.

FIG. 4 illustrates various subsystems of a decentralized computational framework of the clinical trial management system, in accordance with an embodiment.

FIG. 5 illustrates various subsystems of a trial configuration subsystem of the clinical trial management system, in accordance with an embodiment.

FIG. 6 illustrates various subsystems of a real time analytics and monitoring subsystem of the clinical trial management system, in accordance with an embodiment.

FIG. 7 illustrates an interoperability framework communicatively coupled to external systems, in accordance with an embodiment.

FIG. 8 illustrates an exemplary blockchain-configured ecosystem architecture containing one or more components of the clinical trial management system, in accordance with an embodiment.

FIG. 9 illustrates a representative hardware environment for practicing various embodiments.

### DETAILED DESCRIPTION

The embodiments herein and the various features and advantageous details thereof are explained more fully with reference to the non-limiting embodiments that are illustrated in the accompanying drawings and detailed in the following description. Descriptions of well-known components are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

In the following detailed description, reference is made to the accompanying drawings which form a part hereof, and in which is shown by way of illustration specific embodiments in which the embodiments herein may be practiced. These embodiments, which are also referred to herein as “examples,” are described in sufficient detail to enable those skilled in the art to practice the embodiments herein, and it is to be understood that the embodiments may be combined, or that other embodiments may be utilized and that structural, logical, and electrical changes may be made without departing from the scope of the embodiments herein.

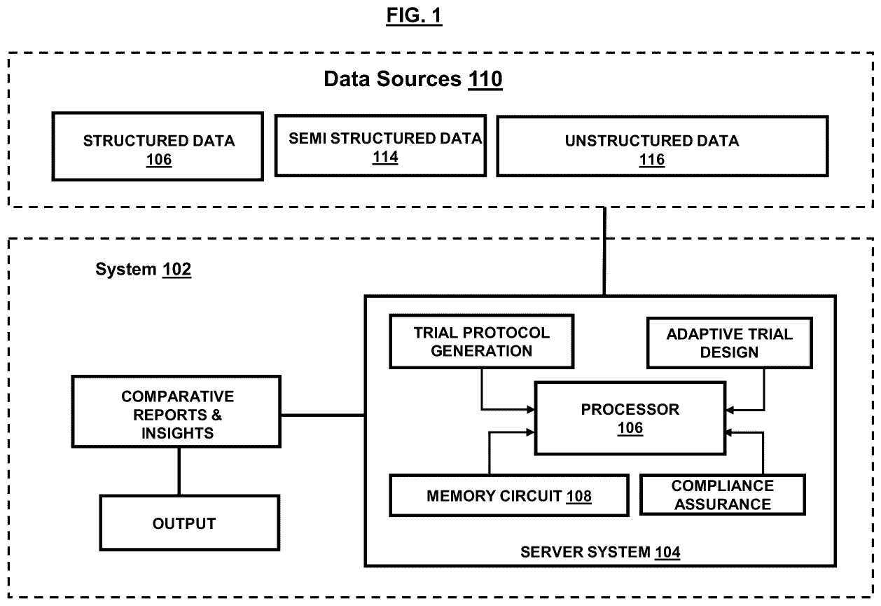

FIG. 1 illustrates an exemplary ecosystem 100 containing a clinical trial management system also referred to as system or CTMS 102 through this document interchangeably without limitations for autonomous design, execution, and analysis of experimental studies. The system 102 may include a server system 104 which may include or be coupled communicatively and/or operatively to one or more processors (also referred to as processor 106). The one or more processors 106 hereafter referred to as the processor 106 for simplicity of description is configured to receive, from a user interface, or generate a computer executable trial protocol. In some embodiments, the one or more processors 106 may receive the trial protocol from a user input, while in other embodiments, the processor 106 may be configured to generate the trial protocol by itself.

The trial protocol, as depicted in FIG. 1, serves as a foundational framework that defines operational parameters or experimental parameters of the experimental study managed by the system 102. The trial protocol may include a structured set of instructions, rules, and conditions that govern various aspects of the study, including but not limited to participant recruitment criteria, intervention types, outcome measures, and trial duration. The trial protocol may be configured to accommodate a wide range of study designs, including randomized controlled trials, observational studies, and adaptive trials, A/B tests and the like without limitations, thereby providing flexibility and scalability to researchers.

In some embodiments, the trial protocol may be dynamically generated by the processor 106 based on multi-dimensional input criteria received from a user or extracted from pre-existing data repositories and stored in a memory circuit 108 by the system 102. These criteria may include demographic, clinical, and behavioral attributes of potential participants, predefined study objectives, and regulatory compliance requirements. The system 102 may further utilize advanced computational models, such as reinforcement learning algorithms as would be discussed later, to autonomously optimize the trial protocol by adjusting experimental parameters in real-time. This optimization process may ensure that the trial protocol aligns with evolving requirements of the study, improves statistical power, and reduces potential biases. In embodiments where the user input is provided, the system 102 may facilitate seamless customization of the trial protocol through the user interface, allowing researchers to define specific experimental parameters while maintaining adherence to regulatory guidelines.

The processor 106 may be further configured to generate an adaptive trial design based on the trial protocol. The adaptive trial design may dynamically adjust the experimental parameters of the study in response to interim results obtained during trial execution. The adaptive trial design may be particularly advantageous in optimizing study outcomes by allowing necessary modifications to be made to the experimental study while it is ongoing. These modifications may include, but are not limited to, alterations to sample size, participant allocation, intervention arms, or data collection protocols. The processor 106 may be configured to execute these adjustments in real-time by analyzing continuous data streams (such as data sources 110) and applying predefined or dynamically generated adjustment rules, thereby ensuring that the experimental study remains robust, efficient, and responsive to emerging insights.

The processor 106 may be further configured to store trial data associated with the experimental study. The trial data may include such as patient-reported outcomes, wearable device metrics, and clinical observations, and the like. The trial data may represent a comprehensive collection of information generated throughout the execution of the experimental study, thereby capturing quantitative and qualitative measures that may be critical to evaluating the study outcomes. The stored trial data may be maintained in a secure, centralized or distributed storage infrastructure operatively coupled to the processor 106, thereby ensuring data integrity, accessibility, and scalability of the system 102.

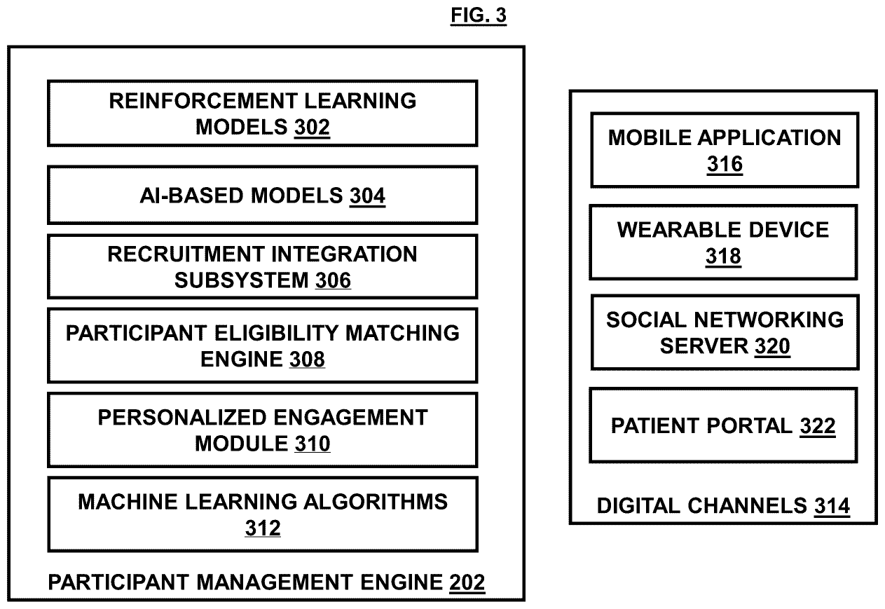

The system 102 may further include a participant management engine 202 configured to facilitate direct-to-consumer patient engagement by enabling patient enrolment and ongoing trial participation through one or more digital channels 314 (as shown in FIG. 3). The participant management engine 202 may interact with the digital channels 314 and associated digital interfaces, such as mobile applications (also referred to as mobile application 316), web portals or patient portals (also referred to as patient portal 322), wearable devices (also referred to as wearable device 318), or social networking platforms (also referred to as social networking server 320), to facilitate recruitment and engagement process. The system 102 ensures that patients can enroll in the experimental study, access trial-related information, and actively participate in trial activities in a user-friendly manner by leveraging the digital channels 314.

The system 102 may be further configured to integrate with one or more patient-associated devices, such as the wearable devices 318, mobile health tools, or software applications, through a decentralized computational framework 204. This integration of the system 102 with various patient-associated devices 412 may allow collection of real-time physiological data from trial participants (or simply participants), such as biometric readings, activity levels, lifestyle and nutrition related parameters or other health metrics, during the execution of the experimental study. The decentralized computational framework 204 may be designed to ensure privacy-preserving data collection and processing, enabling secure, distributed, and efficient handling of participant data. The collected data may then be transmitted to the server system 104, where it may be processed and utilized to generate comparative effectiveness reports that may provide actionable insights on intervention efficacy and safety.

This configuration of the system 102 may allow a high level of engagement with the trial participants while enabling robust data collection from diverse and distributed sources, thereby supporting the scalability and effectiveness of the experimental study conducted through the system 102.

The processor 106 within the server system 104 may be configured to execute a sequence of operations that may begin with the generation or receipt of the trial protocol. In one embodiment, the trial protocol may be generated autonomously by the processor 106 based on the pre-defined multi-dimensional input criteria, including but not limited to participant recruitment criteria, intervention types, outcome measures, and trial duration. Alternatively, the trial protocol may be received from an external system 102, such as a principal investigator's system 102, or inputted by a user through the user interface. The trial protocol may define the foundational blueprint for the experimental study, defining its scope, parameters, and objectives-all executable through computer systems including such as the processor 106.

Once the trial protocol is defined, the processor 106 may generate the adaptive trial design that may enable dynamic adjustments to the study in response to the interim results. This adaptive trial design may allow the experimental study to evolve based on real-time insights, ensuring optimal utilization of resources and adherence to ethical standards. The adaptive design process may include recalibration of participant recruitment strategies, modification of intervention plans, and adjustment of outcome measures, as necessitated by interim analyses.

The processor 106 may further be configured to implement compliance assurance measures. The processor 106 may provide role-specific instructions to trial entities, such as principal investigators, clinical site personnel, and study coordinators. Additionally, the processor 106 may allow automated tracking of compliance with regulatory standards by maintaining audit trails, tracking task completion, and monitoring adherence to predefined guidelines.

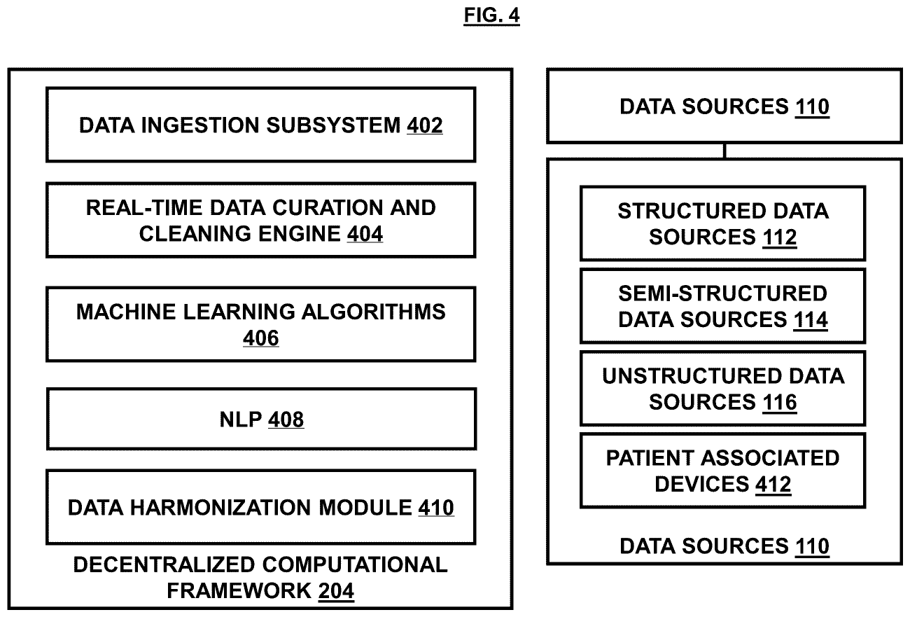

Throughout the execution of the experimental study, the processor 106 may collect and store the trial data, including patient-reported outcomes, wearable device 318 metrics, and clinical observations or other patient associated devices such as collectively referred to and shown as 412 in FIG. 4. This collected data may be ingested and processed through the decentralized computational framework 204, ensuring real-time data integration, privacy preservation, and computational scalability. The server system 104 may analyze the collected data to generate comparative effectiveness reports and insights, which are made accessible to the trial entities such as principal investigators, regulatory bodies, and clinical study sponsors in the form of digitally executable files.

The server system 104 may facilitate direct-to-consumer patient engagement through the participant management engine 202 as will be discussed later. The participant management engine 202 may support patient enrollment, engagement, and participation in the study via multiple digital channels 314, including the mobile applications 316, social networking platforms (also referred to as social networking server 320), and the patient portals 322, and the like without limitations. The system's capability to integrate with the patient-associated devices 412 may allow real-time collection of physiological data or real world data, further improving the robustness of the trial process by generating real world evidence for enrolment, ongoing participation, and for tracking the outcomes.

The system 102 may allow comprehensive execution of the trial process, from protocol generation to the delivery of actionable insights, while maintaining strict compliance with regulatory requirements and ensuring the security and integrity of the trial data. The ecosystem depicted in FIG. 1 exemplifies the integration of advanced computational, communication, and analytical capabilities necessary for conducting autonomous, real-world experimental studies, in an embodiment.

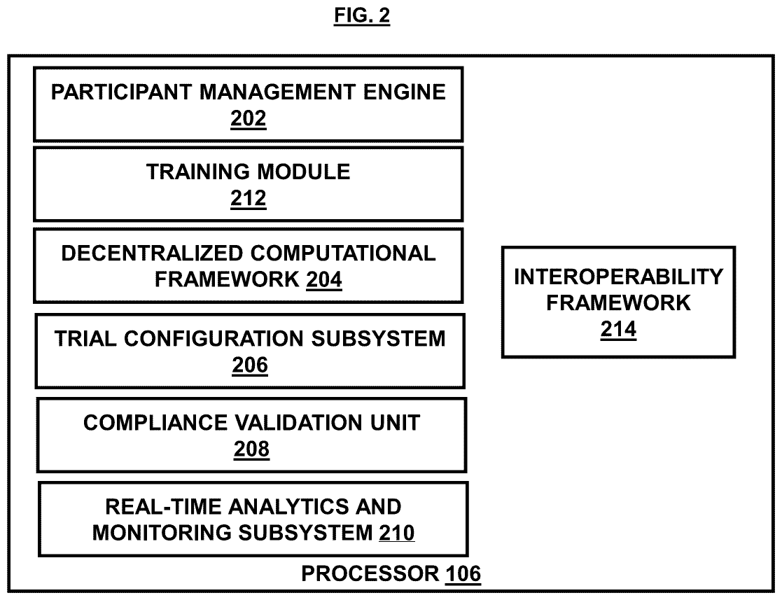

FIG. 2 illustrates various subsystems and modules implemented within the processor 106 of the clinical trial management system (CTMS) described in conjunction with FIG. 1. The processor 106 is configured to enable autonomous design, execution, and analysis of the experimental study as described earlier. The components depicted in FIG. 2 may be operatively and/or communicatively connected to execute tasks in alignment with the trial protocol and the adaptive trial design. Each subsystem or module may be designed to perform specific operations, as discussed below.

The processor 106 may include the participation management engine (also referred to as the participant management engine 202), the decentralized computational module (also referred to as decentralized computational framework 204), a trial configuration subsystem 206, a compliance validation unit 208, a real-time analytics and monitoring subsystem 210, and a training module 212.

The participant management engine 202 may be configured to execute participant engagement, recruitment, and eligibility screening, and the like. FIG. 3 illustrates various subsystems of the participation management engine 202. The participation management engine 202 is discussed herein with respect to FIGS. 1-3. In various embodiments, the participation management engine 202 may facilitate direct-to-consumer patient engagement by enabling patient enrolment and ongoing trial participation through the digital channels 314.

The participant management engine 202 may further include a recruitment integration subsystem 306 and a participant eligibility matching engine. The recruitment integration subsystem 306 may interface with the plurality of digital channels 314, including the mobile applications 316, wearable devices 318, social networking servers 320, and the patient portals 322, to facilitate participant recruitment. The participant eligibility matching engine may automatically classify and match the participants to the experimental study using predefined inclusion and exclusion criteria or any other predetermined criterial based on such as a probabilistic scoring model or the like approaches.

The participant management engine 202 may be configured to facilitate participant recruitment and eligibility screening using one or more AI-based models 304. The participant management engine 202 may be configured to obtain and store tamper-proof informed consent records about the participants through a secure blockchain ledger. The blockchain ledger will be discussed later in the document.

The participant eligibility matching engine 308 may leverage the AI-based models 304 including advanced probabilistic scoring models, such as Bayesian networks or logistic regression, to evaluate a participant's suitability for inclusion in the experimental study based on the predefined inclusion and exclusion criteria or other criteria. These models may consider a wide array of data inputs, including demographic details, medical history, previous treatments, and real-time sensor data from the wearable devices 318, to generate a risk score for each potential participant. For example, the participant eligibility matching engine 308 may analyze data such as age, comorbidities, and genetic predispositions to determine whether a participant aligns with the specific criteria of the study, such as being within a target age range or having a particular condition.

The eligibility matching process may also be supported by machine learning algorithms 312, which may continually improve the accuracy of participant classification through feedback loops. For instance, when a participant is successfully recruited, their health data and other characteristics may be used to refine the AI-based models 304, allowing the participant eligibility matching engine 308 to better predict eligibility for future participants. The participant eligibility matching engine 308 may further incorporate unsupervised learning techniques, such as clustering algorithms, to identify patterns in participant profiles that may not be explicitly captured by predefined criteria, thereby allowing the engine to recommend those participants who may meet latent eligibility conditions not initially considered in the design of the experimental study.

The participant eligibility matching engine 308 may be integrated or communicatively coupled with the recruitment integration subsystem 306, thereby ensuring that only eligible participants are presented with the study enrollment opportunities. The participant eligibility matching engine 308 may utilize real-time data updates, so that any changes in participant health status, treatment history, or other critical factors may be immediately reflected in their eligibility status. This dynamic approach may allow the system 102 to continuously adapt to evolving participant data throughout the trial, enhancing both the accuracy and efficiency of participant selection and continuation for the experimental study in a continuously controlled and managed format.

The participant management engine 202 may further include a personalized engagement module 310, which may be communicatively linked to the recruitment integration subsystem 306. The personalized engagement module 310 may be configured to deliver context-aware messaging and tailored recruitment campaigns through dynamic content rendering on the one or more digital channels 314. The personalized engagement module 310 may monitor and adapt participant engagement strategies using reinforcement learning models 302 based on user interaction patterns.

The personalized engagement module 310 may further improve participant retention and satisfaction by providing individualized support throughout the experimental study. For instance, the personalized engagement module 310 may utilize real-time data analysis to identify those participants who may require additional guidance or encouragement to remain active in the study with the use of a real-time analytics and monitoring subsystem 210 and training module 212 as illustrated in FIG. 2. The personalized engagement module 310 may deliver customized reminders, motivational content, or step-by-step instructions specific to the participant's stage in the trial based on this analysis. Additionally, the personalized engagement module 310 may generate feedback loops by collecting user responses to engagement efforts, allowing the participant management engine 202 to continuously refine messaging strategies and improve overall participant experience. This tailored approach allows participants to feel supported and valued, thereby fostering higher levels of adherence and trust in the experimental study process.

The participant management engine 202 may leverage the reinforcement learning models 302 to optimize participant engagement and recruitment strategies dynamically. These models may analyze the user interaction patterns and the trial data to identify the most effective communication approaches for individual participants or groups. Through continuous learning from participant behavior, the reinforcement learning models 302 may adapt messaging, campaign timing, and content delivery to maximize engagement outcomes. For example, the models may determine optimal notification frequencies or preferred communication channels based on past responses, enabling the system to personalize interactions further. This iterative learning process allows that engagement strategies remain effective and responsive to changing participant needs throughout the experimental study.

As discussed above, the participant management engine 202 may be configured to facilitate direct-to-consumer engagement by enabling patient enrolment and ongoing trial participation through the digital channels 314, wherein the digital channels 314 can be one of a mobile device or application or wearable device or any other associated and networked device of the potential participant (or participant) as has been discussed above.

The direct-to-consumer patient engagement mechanism, implemented via the participant management engine 202, may provide an innovative approach for participant enrollment and trial participation in the experimental study. This may eliminate the need for traditional, resource-intensive survey-based or other manual ways of recruitment and enable seamless, proactive enrollment through the integration of patient-associated devices and digital channels 314 automatically.

The participant management engine 202 may be configured to facilitate touch-free setup for automated onboarding and connectivity, allowing potential participants to register and link their devices (such as mobile phone or wearables etc) called participant-associated devices to the system 102 with minimal setup. These devices may include wearable health trackers, mobile health applications, smartwatches, or other IoT-enabled health monitoring tools. Upon connection, the system may automatically establish secure, continuous communication with the device, leveraging standard APIs and device integration protocols.

In an exemplary embodiment, the participant management engine 202 may dynamically detect and authenticate the participant-associated devices. For instance, a participant using a wearable fitness tracker may register with the system and allow synchronization, allowing the system to automatically ingest real-time physiological data, such as heart rate, activity levels, sleep patterns, or blood glucose levels or other form of real-world data. The automated onboarding may allow device compatibility and data integrity using predefined standards, such as HL7 or FHIR, to enable uniform data processing.

Once the participant's device is registered and synced, the participant management engine 202 may continuously monitor and process the real-time physiological data or real-world data transmitted through the decentralized computational framework 204. This real-time data ingestion pipeline may be powered by machine learning algorithms 406 that may analyze and classify the incoming data against predefined criteria stored in a trial eligibility database or the memory circuit 108.

For example, the system 102 may track a participant's daily activity levels and identify trends, such as irregular heart rhythms or specific health conditions, that match the inclusion criteria of an experimental study. The system 102 may employ predictive modeling and probabilistic scoring to proactively identify participants with the highest likelihood of meeting a study's requirements.

Upon identifying a suitable experimental study, the participant management engine 202 may autonomously generate a personalized enrollment invitation, which may be delivered to the participant via the one or more digital channels 314, such as a mobile app notification, SMS, or email. The invitation may include details of the study, potential benefits, risks, and a direct link to accept or decline the invitation in the form of a computer executable instruction.

To streamline the process further, the participant management engine 202 may integrate with the compliance validation unit 208 to automatically verify the participant's informed consent and regulatory compliance before finalizing the enrollment. For example, the system 102 may send the participant a multimedia consent form that they can review and digitally sign within the mobile application 316, with the signed record stored securely on a blockchain-enabled ledger for audit purposes.

In addition to proactive enrollment, the system 102 may allow ongoing participant engagement throughout the trial by providing real-time feedback and progress tracking via digital interfaces as discussed elsewhere in the document. The participants may view summaries of their data contributions, receive tailored health tips based on trial objectives, and communicate with trial administrators through in-app messaging features. This kind of adaptive engagement powered by reinforcement learning models 302 may allow the participant management engine 202 to dynamically adjust frequency and content of interactions to maximize participant retention and adherence to trial the protocol.

This automated onboarding approach may transform participant recruitment into a seamless, automated process, enhancing accessibility and inclusivity for direct-to-consumer engagement while optimizing trial efficiency and data quality. By reducing the reliance on manual interventions and traditional manual processes, the system 102 may accelerate participant enrollment and ensure a higher match rate for experimental studies.

In various embodiments, the system 100 may allow autonomous direct-to-consumer recruitment of the participants for digitally executed experimental studies such as the experimental study discussed above. The processor 106 may be configured to receive, from the user or a possible prospective participant via the user interface, the participant data in a computer-executable format. The participant data may include participant-specific characteristics such as demographic information, medical history, and configuration details of an associated device generating the real-world data. The processor 106 may synchronize with the associated device such as the associated device 412 to automatically gather the real-world data from the user device. The processor 106 stores the participant data and continuously updates the real-world data of the user in a participant database such as the memory circuit 108. The processor 106 may execute the machine learning algorithms 312 to analyze the participant data and continuously assess eligibility against the trial protocols associated with the experimental studies. The processor 106 may match the user to eligible studies when the stored data satisfies at least one of the trial protocols corresponding to certain matched studies.

The processor 106 may be further configured to either receive or generate the plurality of trial protocols defining such as study-specific recruitment criteria, intervention details, outcome measures, and inclusion and exclusion parameters for the digitally executed experimental studies. The processor 106 may be further configured to generate and transmit an automated digital invitation to a matched user, for enrolment into a matched experimental study upon affirmative response. The processor 106 may be further configured to automatically onboard the user for the matched experimental study. The processor 106 may obtain one or more tamper-proof informed consent records from the user using a blockchain-enabled data assurance device as discussed later in the document. The processor 106 may be configured to initiate a real-time synchronization and update of the associated device to collect and transmit the real-world data including such as computer executable physiological data, computer executable nutrition data, and computer executable lifestyle data of the user for use in the matched experimental study without limitations. As discussed, in some embodiments, the user device may be a wearable device or a mobile application or a patient portal etc generating the real world data. In some embodiments, the user device may be a mobile application generating the real world data. In some embodiments, the processor 106 may be configured to enroll the same user in more than one experimental studies simultaneously or at different times such that the user may participate in more than one experimental studies depending on matching based on the trial protocols associated with the experimental studies to conduct A/B test for multiple interventions. In an embodiment, the automatic onboarding may include obtaining one or more tamper-proof informed consent records from the user using a blockchain-enabled data assurance device as will be discussed later. The process of initiating a real-time synchronization of the associated device 412, with the server system 104 may be enabled to collect and transmit the real-world data including the physiological data of the user and/or data from the mobile application or any other associated device for use in the matched experimental study. The system 102 may continuously update the participant database such as the or communicatively connected to the memory circuit 108 without limitations with newly ingested data from the associated device 412 or any other data source of the user at regular intervals, enabling reusability of participant profiles for subsequent studies. The system 102 may autonomously adapt the recruitment by updating the machine learning algorithms 312 based on historical participant engagement and recruitment outcomes.

The processor 106 further includes the decentralized computational framework 204 shown in FIGS. 2 and 4, that may be configured to integrate the heterogeneous data streams or data sources 110 that may include the real world data and capable of generating real world evidence originating from the plurality of sources 110, including structured (also referred to as structured data sources 112), unstructured data (also referred to as unstructured data sources 116), and semi-structured data sources 114 located at remote locations and associated with the one or more participants. The decentralized computational framework 204 is discussed herein in conjunction with FIGS. 1-4.

The decentralized computational framework 204 may include a data ingestion subsystem 402 for privacy-preserving integration of data from the structured data sources 112, unstructured data sources 116, and the semi-structured data sources 114. The decentralized computational framework 204 may further include a real-time data curation and cleaning engine 404 utilizing one or more machine learning algorithms 406 to identify, correct, and harmonize inconsistencies within the ingested data.

The data ingestion subsystem 402 may employ advanced cryptographic protocols, such as homomorphic encryption or differential privacy techniques, to ensure privacy-preserving data integration. The data ingestion subsystem 402 may perform data normalization that may translate the heterogeneous data streams into a unified format, thereby enhancing interoperability across multiple data streams. The data ingestion subsystem 402 may also perform metadata tagging that may assign descriptive tags to the incoming data streams 110, enabling efficient indexing and retrieval within the decentralized computational framework 204.

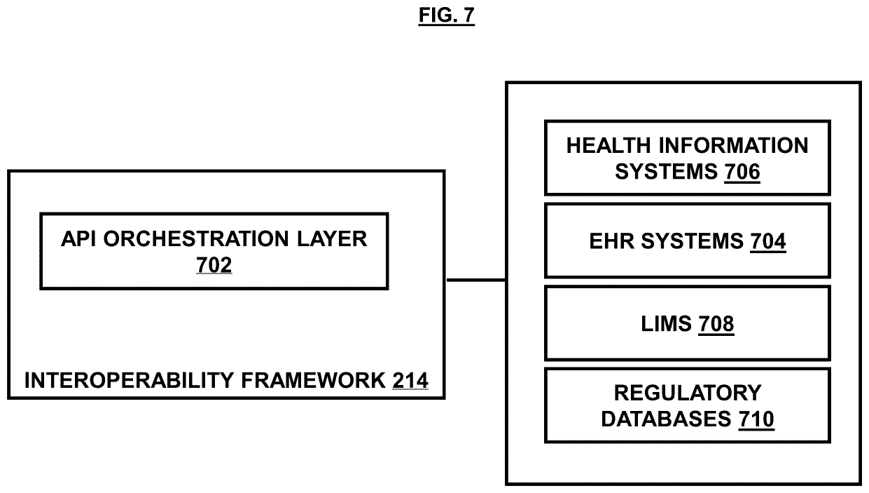

The data ingestion subsystem 402 within the decentralized computational framework 204 may be designed to handle and process diverse types of real-world data that may be critical for the experimental study. As discussed above, these data streams 110 may include the structured data 112 from such as electronic health records (EHRs) 704 shown in FIG. 7, clinical trial databases, laboratory information management systems (LIMS) 708, and other structured formats such as CSV or SQL databases; the unstructured data sources 116 from such as clinical notes, medical literature, patient feedback surveys, audio or video recordings of clinical interactions, and imaging data (such as radiology or pathology images); and the semi-structured data sources 114 originating from such as the wearable devices 318, sensor data from IoT-enabled health equipment, and mobile health applications or patient input devices or the patient associated devices 412, and the like. In real-world scenarios, the system 102 may need to ingest large volumes of data generated across multiple touchpoints within the experimental study, such as real-time physiological readings from the wearable devices 318 (e.g., heart rate, blood pressure, or glucose levels), responses to survey-based outcome measures (such as symptom tracking or quality of life assessments), and direct clinical observations made by healthcare providers (e.g., diagnosis, treatment history, or test results). The data ingestion subsystem 402 may therefore be equipped to handle not only the structured data 112 from clinical systems but also the heterogeneous, the unstructured 116, and the semi-structured data 114 that may emerge from diverse patient interactions and digital health technologies in rea world scenarios.

To facilitate privacy-preserving data integration, the ingestion subsystem (also referred to as data ingestion subsystem 402) may employ cryptographic techniques. For example, homomorphic encryption may be utilized to enable computations on encrypted data, allowing for data processing without exposing sensitive patient information. In embodiments, differential privacy algorithms may be applied to anonymize data points while maintaining statistical integrity necessary for clinical analyses. As the real-world clinical data may be inherently noisy and incomplete, the data ingestion subsystem 402 may integrate intelligent preprocessing methods to identify, filter, and securely collect relevant data streams, ensuring that any sensitive data is kept secure and compliant with regulations such as HIPAA or GDPR.

The data ingestion subsystem 402 may be further designed to utilize advanced data pipeline architectures such as Apache Kafka or Apache Nifi, allowing for real-time ingestion of data from the distributed sources 110. Data pipelines may be designed to be fault-tolerant, capable of handling both high-throughput data streams (such as continuous monitoring data from the wearable devices 318) and large-batch data processing (such as periodic lab reports or patient visit records). The system 102 may ensure that all data, irrespective of its origin or format, is properly ingested, time-stamped, and stored in a centralized data warehouse contained within the memory circuit 108 for further processing.

The data ingestion subsystem 402 may include mechanisms to handle various data quality issues such as missing or inconsistent data entries, duplicate records, and discrepancies between the different data sources 110. For example, if a participant's blood pressure readings from the wearable device 318 are inconsistent with clinical measurements, the data ingestion system 402 may flag this as an anomaly, allowing it to be addressed by downstream cleaning and curation processes. The data ingestion subsystem 402 may use the machine learning models 406 to predict missing values or infer missing measurements from correlated data streams (e.g., estimating a participant's activity level from sensor data when direct measurements are unavailable).

The data ingestion subsystem 402 may ensure that all data, regardless of type or source, is securely and efficiently integrated into the decentralized computational framework 204, providing a reliable foundation for real-time data analysis, adaptive trial design, and accurate reporting of clinical outcomes in the experimental study. Using advanced privacy-preserving techniques and the data integration protocols, the data ingestion subsystem 402 may support seamless flow of data throughout the system 102 while safeguarding patient privacy and maintaining regulatory compliance.

The real-time data curation and cleaning engine 404, operatively coupled to the data ingestion subsystem 402, may utilize a multi-stage pipeline comprising the machine learning algorithms 406 to identify, correct, and harmonize inconsistencies in the ingested data. The curation process may begin with anomaly detection algorithms, such as unsupervised clustering models or statistical outlier detection, to flag and isolate deviations from expected data patterns. Subsequently, the real-time data curation and cleaning engine 404 may apply probabilistic methods or neural network-based models to fill missing values in the data. The real-time data curation and cleaning engine 404 may further leverage natural language processing (NLP) 408 techniques to preprocess the unstructured data 116 by removing noise, normalizing text, and extracting relevant features.

In embodiments, the real-time data curation and cleaning engine 404 may perform schema mapping to align the incoming data streams 110 with predefined schema templates, ensuring compliance with system-specific data standards. The real-time data curation and cleaning engine 404 may also include a data harmonization module 410 that may employ entity resolution algorithms, such as probabilistic record linkage or graph-based matching, to consolidate redundant or duplicate entries. The real-time data curation and cleaning engine 404 may perform these operations in real-time, so that the ingested data is not only consistent and accurate but also optimally prepared for subsequent analytics, machine learning model training, and adaptive trial adjustments within the experimental study. These technical advancements may collectively ensure that the data ingestion and cleaning processes are robust, scalable, and aligned with regulatory compliance requirements, effectively addressing the challenges of integrating and processing the diverse data streams 110 in a decentralized computational environment.

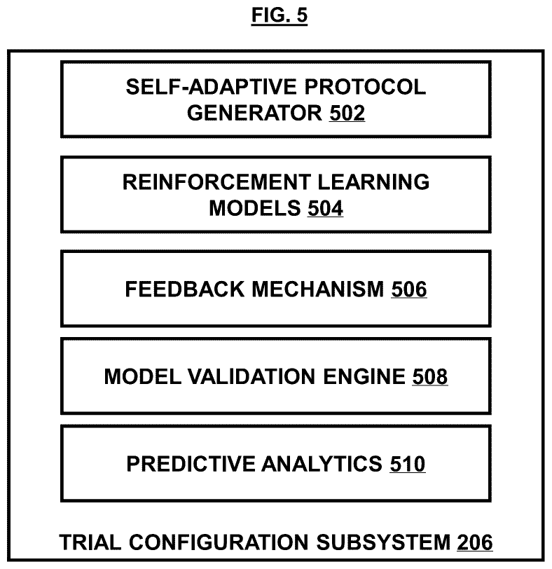

FIG. 5 illustrates various components of the trial configuration subsystem 206. The trial configuration subsystem 206 is discussed herein in conjunction with FIGS. 1-5. As shown in FIG. 5, the trial configuration module or subsystem 206 may include a self-adaptive protocol generator 502 utilizing one or more reinforcement learning models 504 to autonomously define and optimize experimental parameters based on multi-dimensional input criteria and generate the computer executable trial protocol.

The self-adaptive protocol generator 502 may utilize one or more reinforcement learning models 504, such as policy gradient methods, Q-learning, or actor-critic algorithms, to autonomously determine optimal configurations for the computer executable trial protocol. The reinforcement learning models 504 may be trained on historical trial data, simulated environments, or both, to identify experimental designs that maximize study efficiency, compliance, and outcome reliability. The self-adaptive protocol generator 502 may be operatively coupled to the decentralized computational framework 204 and the participant management engine 202 to acquire multi-dimensional input criteria, including participant characteristics, study objectives, intervention types, and anticipated outcome measures. The input criteria may further include constraints such as ethical guidelines, regulatory requirements, and available resources. The reinforcement learning models 504 of the trial configuration subsystem 206 may analyze the input criteria in real time, dynamically adjusting trial parameters, such as sample size, randomization strategy, or data collection frequency, to ensure alignment with the study objectives while adhering to operational constraints.

In some embodiments, the self-adaptive protocol generator 502 may employ a feedback mechanism 506, wherein interim results from the ongoing experimental study are analyzed to refine the trial protocol. The feedback mechanism 506 may incorporate real-time data from the decentralized computational framework 204, such as the wearable device 318 metrics and patient-reported outcomes, to evaluate the efficacy and safety of the interventions. Based on this evaluation, the self-adaptive protocol generator 502 may modify the experimental parameters, such as intervention dosage or the inclusion of additional participant cohorts, thereby enabling an adaptive trial design.

The self-adaptive protocol generator 502 may further include a model validation engine 508 to ensure reliability and accuracy of the reinforcement learning models 504 used in protocol optimization. The model validation engine 508 may execute cross-validation processes and deploy predictive analytics 510 to verify that the generated protocol meet predefined performance metrics. In some embodiments, the trial configuration subsystem 206 may integrate with the compliance validation unit 208 to ensure that any changes to the trial protocol remain compliant with computer executable predefined guidelines such as regulatory standards and ethical guidelines. This integration may be achieved through automated cross-referencing of updates of the trial protocol against stored compliance rules and regulations, thereby providing an end-to-end solution for autonomous trial configuration without any manual intervention of experts such as principal investigators. This way, the compliance validation unit 208 may be configured to enforce real-time adherence to the computer executable predefined guidelines.

The compliance validation unit 208 may be configured to continuously monitor updates to the trial protocol and verify their adherence to the computer executable predefined guidelines. These predefined guidelines may include regulatory frameworks, ethical standards, data integrity requirements, and operational constraints relevant to the experimental study. The compliance validation unit 208 may incorporate a rule-based inference process that may systematically apply the compliance rules to evaluate protocol modifications in real time. In some embodiments, the compliance validation unit 208 may utilize natural language processing algorithms to parse and interpret textual descriptions of regulatory and ethical standards, so as to expand its capability to dynamically accommodate updates in compliance frameworks without requiring manual reprogramming.

The compliance validation unit 208 may further include a real-time auditing process to ensure transparency and accountability throughout the trial configuration and execution process. The real-time auditing may log all changes to the trial protocol, including rationale behind each modification and corresponding compliance validation results. These logs may be stored on a secure, immutable ledger, such as a blockchain, to create a tamper-proof audit trail. The compliance validation unit 208 may provide automated notifications to relevant stakeholders, such as trial administrators and regulatory bodies, whenever a compliance violation is detected or when updates are made to the compliance rules. This proactive notification mechanism may allow timely corrective actions and may minimize risks associated with non-compliance, thereby enhancing reliability and ethical robustness of the experimental study.

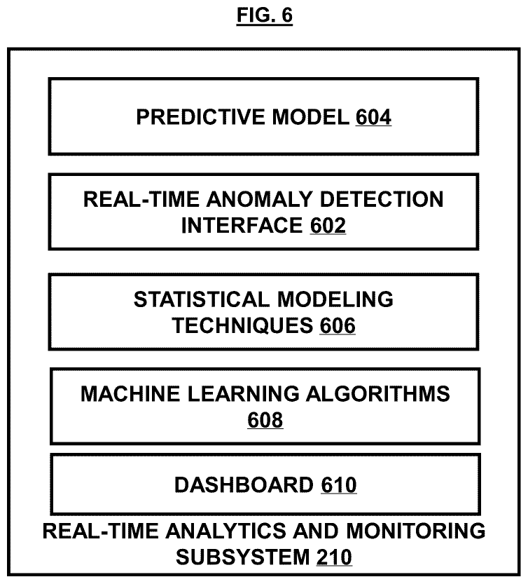

FIG. 6 illustrates various components of the real-time analytics and monitoring subsystem 210, in accordance with some embodiments. The real-time analytics and monitoring subsystem 210 is discussed herein in conjunction with FIGS. 1-6. The real-time analytics and monitoring subsystem 210 may be communicatively coupled to the decentralized computational framework 204 and the participants management engine (also referred to as participant management engine 202).

The real-time analytics and monitoring subsystem 210, as described in FIGS. 1 and 6, may be configured to generate actionable insights into participant health, study outcomes, and protocol efficacy by analyzing the ingested data streams in real-time obtained by the decentralized computational framework 204. The real-time analytics and monitoring subsystem 210 may include a predictive model 604 designed to assess safety and efficacy of the intervention by continuously integrating the data streams or the data sources 110, such as patient-reported outcomes, wearable device metrics, and clinical observations. The predictive model 604 may employ advanced machine learning algorithms 608, including supervised and semi-supervised learning techniques, to evaluate historical and real-time data patterns. The predictive model 604 may further generate probabilistic risk scores to anticipate adverse events or deviations from expected trial outcomes, thereby enabling preemptive mitigation strategies and stop or adjust the experimental study accordingly.

The real-time analytics and monitoring subsystem 210 may include a real-time anomaly detection interface 602 operatively coupled to the predictive model 604. The real-time anomaly detection interface 602 may utilize advanced statistical modeling techniques 606 and the machine learning algorithms 608 to identify deviations from expected trends within the data streams 110. These techniques may include clustering, density estimation, and time-series analysis to detect the deviations or anomalies indicative of potential adverse events or protocol inefficiencies. Upon identifying such anomalies, the real-time anomaly detection interface 602 may automatically generate visual or text alerts displayed on a dashboard. The dashboard may present the alerts in a user-friendly format, including graphical visualizations such as trend charts, heat maps, or threshold markers, to facilitate rapid interpretation by the trial administrator.

In some embodiments, the real-time anomaly detection interface 602 may trigger automated notifications to a trial administrator system upon detecting significant deviations. These notifications may include details about the nature of the anomaly, the affected participant(s), and a recommended course of action based on an analysis by the predictive model 604. The dashboard 610 may provide interactive features that may allow the trial administrator to investigate the anomalies further, enabling real-time decision-making and intervention to ensure participant safety and study integrity. The real-time analytics and monitoring subsystem 210 may ensure that the experimental study remains adaptive, safe, and aligned with its predefined objectives by employing these advanced capabilities.

The system 102 may further include the interoperability framework 214. The interoperability framework 214, as shown in FIGS. 2 and 7, may facilitates seamless data exchange between the various components of the system 102 and external health information systems 706, such as electronic health record (EHR) systems 704, laboratory information management systems (LIMS) 708, and regulatory databases 710. The interoperability framework 214 may include an API orchestration layer 702 configured to implement FHIR (Fast Healthcare Interoperability Resources) specifications, enabling standardized, bidirectional communication. In some embodiments, the API orchestration layer 702 may also ensure that data related to the anomalies, insights, or interventions generated by the real-time analytics and monitoring subsystem 210 may be securely shared with certain defined external systems in compliance with applicable regulatory and operational standards. This integration may support cross-platform interoperability, further enhancing the utility and robustness of the system across diverse distributed environments.

In some embodiments, the system 102 may facilitate compliance assurance, as described herein, using the training module 212 and the compliance validation unit 208. The compliance validation unit 208 may be configured to enforce regulatory compliance by delivering one or more role-specific instructions and tracking compliance activities in real-time during an execution of the experimental study through the training module 212.

The training module 212 may be configured to generate and disseminate dynamic instructional content to users based on their assigned roles in the experimental study. The roles may include, but are not limited to, principal investigators, clinical coordinators, data analysts, and trial participants. The training module 212 may classify each user into respective predefined roles, leveraging user profile metadata stored within the system.

In an exemplary embodiment, the instructional content may be generated in accordance with regulatory standards, including but not limited to, FDA 21 CFR Part 11, ICH-GCP guidelines, or HIPAA requirements. The training module 212 may customize the instructional content based on a combination of user roles and contextual factors, such as the trial protocol, geographic regulatory requirements, or study phase. For example, a trial administrator may receive interactive modules detailing data integrity protocols, while participants may receive multimedia consent forms outlining trial expectations and risks.

The training module 212 may deliver the instructional content through multiple digital channels 314, including web portals, mobile applications (also referred to as mobile application 316), and email notifications. Each instruction of the instructional content may include embedded verification mechanisms, such as digital acknowledgment signatures or quiz-based assessments, to confirm that the user has understood and accepted the guidance provided.

The training module 212 may further be configured to monitor and log user interactions with the instructional content. The training module 212 may employ machine-readable logging mechanisms to automatically document timestamps, completion statuses, and acknowledgment records associated with each instruction delivered. The compliance validation unit 208 and/or the training module 212 may implement periodic audit checks to validate that the user has met specific compliance milestones, such as completing required training modules (also referred to as training module 212) before participating in a study phase.

The training module 212 and the compliance validation unit 208 may be integrated with a centralized compliance dashboard to provide a real-time overview of compliance activities across all users. The dashboard 610 may utilize visualization tools, such as heat maps or progress bars, to highlight non-compliant users or delayed activities. The compliance validation unit 208 may trigger automated notifications or escalation protocols in response to non-compliance, ensuring that corrective actions are initiated promptly.

The training module 212 may operate in conjunction with other system components, such as the participant management engine 202 and the decentralized computational framework 204 and the compliance validation unit 208, to provide seamless compliance management. The training module 212 and the compliance validation unit 208 may interface with the participant management engine 202 to verify that the participants have completed informed consent training before enrollment. Similarly, the training module 212 and the compliance validation unit 208 may leverage the decentralized computational framework 204 to secure and validate compliance logs using blockchain-based tamper-proof mechanisms.

In some embodiments, the compliance validation unit 208 may implement machine learning algorithms 608 to predict potential compliance risks based on historical user behavior. For example, the compliance validation unit 208 and/or the training module 212 may analyze patterns of delayed instructional completions and proactively alert administrators of users likely to breach compliance deadlines.

Through delivery of role-specific instructions and employing automated tracking mechanisms, the training module 212 and the compliance validation unit 208 may provide robust compliance assurance with regulatory standards, reducing the need for manual oversight and enhancing the reliability of the experimental study conducted across distributed environments.

In various embodiments, a method is provided that facilitates autonomous design, execution, and analysis of the real-world data-driven experimental study conducted across the distributed environments. The method may include receiving, through the user interface, or generating the computer-executable trial protocol. The trial protocol may encompass at least one of the participant recruitment criteria, intervention types, outcome measures, and trial duration. Based on the received or generated trial protocol, the method may further involve generating the adaptive trial design. The adaptive trial design may incorporate one or more dynamic adjustments to the experimental study, informed by the one or more interim results obtained during the study.

The method may include collecting the trial data from the one or more trial participants through the decentralized computational framework 204. This framework may be integrated with the one or more patient-associated devices, enabling the acquisition of the trial data in a computer-executable format. The data may include the real-time physiological measurements, participant-reported outcomes, wearable device 318 metrics, and clinical observations. The collected trial data may then be stored in the data repository or the memory circuit 108 for subsequent analysis and reporting.

The method may facilitate direct-to-consumer enrollment and ongoing trial participation through the participant management engine 202 to improve participant engagement. This may be achieved via the one or more digital channels 314, streamlining access for the participants and promoting sustained involvement in the study. The trial data may be analyzed to generate comparative effectiveness reports, offering insights into the relative performance of various interventions.

The method may allow compliance with one or more regulatory standards through the training module 212. The training module 212 may be configured to deliver the role-specific instructions to the participants and other study personnel based on their assigned roles within the experimental study. The training module 212 and/or the compliance validation unit 208 may track compliance activities through automated logging and monitoring of user interactions, thereby ensuring adherence to regulatory requirements throughout the study lifecycle.

In an embodiment, the system 102 may be configured as or be integrated with an adaptive software-as-a-service (SaaS)-based system similar to the system 102 for autonomously managing the experimental study using the real-world data. In such embodiments, the system 102 may be configured generate or receive one or more trial protocols such as the trial protocol defining participant criteria, one or more interventions such as the interventions, one or more outcomes such as the study outcomes discussed above, and respective durations. The system 102 may be configured to facilitate the recruitment of the participants (or users) via the one or more digital channels 314 based on assessment of their eligibility against the participant criteria. The system 102 may continuously analyze the real-world data associated with the participants for the adaptive trial adjustments, anomaly detection, and outcome generation.

The system 102 may be configured to refine the protocol using the machine learning algorithms 608 based on the interim results of the experimental study. The recruitment may be performed dynamically by assessing the eligibility using the real-time physiological data or other forms of real-world data and probabilistic models. In various embodiments, the one or more digital channels 314 may include such as the mobile application 316, wearable device 318, and a social networking server 320, and the like without limitations. The system 102 may be further configured to enroll the one or more participants based on the participant criteria.

In an embodiment, the experimental study may be a digitally conducted A/B test such that the interventions may include at least two interventions and, and both interventions may be delivered to same participants so as to allow conducting the A/B test. The system 102 may be configured to generate outcomes for both the interventions to generate a computer executable comparative report which may indicate efficacy or comparative effectiveness of the interventions thereby facilitating the A/B tests.

In embodiments, the system 102 or the processor 106 may be configured to implement a controlled comparative testing mechanism by allocating at least some of the participants into at least two groups, wherein each group receives distinct interventions such as the interventions defined in the trial protocols. The system 102 may collect and analyze the real-world data from the participants in each group, including the outcomes and the real-world data comprising the physiological parameters, to evaluate the comparative effectiveness of the distinct interventions. In an embodiment, the system 102 may generate the adaptive trial protocols based on an analysis from the at least two groups to optimize an intervention efficacy.

In an embodiment, the system 102 may be configured to either receive, from the user interface, the trial protocol specifying one or more of the participant recruitment criteria, intervention types, outcome measures, and the trial durations, or autonomously generate the trial protocol by applying the machine learning algorithms to analyze the historical study data and the multi-dimensional input criteria.

The system 102 may facilitate the recruitment of the participants by integrating with the digital channels 314, including such as the mobile application, the wearable device, and the social networking server, and the like without limitations. The system 102 may dynamically assess participant eligibility by processing real-time physiological measurements and participant-reported inputs. The system 102 may automatically enroll the into the experimental study when their data satisfies the participant recruitment criteria specified in the trial protocol. In an embodiment, the system 102 may execute computational models for analyzing the real-world data during the experimental study to generate the trial outcomes.

In some embodiments, the system 102 may be configured to apply predictive models to monitor safety and efficacy of the interventions, detect the anomalies, and predict the adverse events.

In various embodiments, a method is provided for autonomously managing the experimental study using the real-world data (including but not limited to the physiological data) through the adaptive software-as-a-service (SaaS)-based system 102. The method may include generating or receiving the trial protocol. As discussed above, the trial protocol may specify the participant criteria, interventions, outcome or outcome measures, and duration of the experimental study. The method may include assessing the eligibility of the participants against the participant criteria specified in the trial protocol. The method may include facilitating the recruitment of the participants through the digital channels 314. The method may include continuously analyzing the real-world data associated with the participants during the experimental study to perform such as the adaptive trial adjustments, detecting the anomalies, and generating or recording the outcomes measures. The analysis may be conducted using the computational models configured for real-time data processing and decision-making.

In some embodiments, the method may allow adjusting the trial protocol based on the anomalies and the adverse events. The real-time insights and the trial outcomes may be presented through the dashboard.

In an embodiment, the recruitment of the participants may be performed dynamically by assessing the eligibility using at least one of the real-time physiological data and probabilistic models.

In various embodiments, the method may include implementing the controlled comparative testing mechanism by allocating the participants into at least two groups, such that each group receives distinct interventions defined in the trial protocols. Accordingly, the method may then include collecting and analyzing the real-world data from participants in each group, including the outcomes and the real-world time comprising the physiological parameters, to evaluate the comparative effectiveness of the distinct interventions. Subsequently, the method may allow generating or updating the adaptive trial protocol based on an analysis from the two groups to optimize an intervention efficacy that provides an outcome through the study or the A/B test conducted in a digitally controlled manner.

In an embodiment the system 102 may automatically allocate the participants to the experimental study by matching their eligibility such that the allocation includes multi-group intervention comparisons to facilitate the A/B tests. This allows the same participants to be part of multiple interventions so as to compare effectiveness of the multiple interventions in the same groups.

Embodiments of the disclosed invention may facilitate a consumer-centric marketplace system that may be designed to allow participation in real-world comparative effectiveness studies such as the experimental studies for healthcare products. In an exemplary implementation, the system 102 may focus on vitamins, supplements, and other non-traditional healthcare services, and the like without limitations.

The marketplace system (not shown) may leverage the system 102 to enable consumer-driven comparative effectiveness studies. This may allow consumers to participate in A/B testing of healthcare products, thereby generating real-world health metrics and real-world evidence. The collected metrics may facilitate an unbiased and data-driven evaluation of product effectiveness.

The system 102 may include artificial intelligence (AI)-powered engagement modules configured to interact with the consumers regularly. This may allow collecting data on health outcomes, side effects, and consumer satisfaction, ensuring that insights are actionable and accurately reflect user experiences. Such continuous data collection improves relevance of generated insights during and after the experimental studies.

To ensure transparency and data integrity, the system 102 may employ the blockchain technology to record and verify consumer feedback. The blockchain may create an immutable ledger, guaranteeing that insights presented on the marketplace system are genuine and free from vendor manipulation. This may foster trust and credibility within the marketplace by ensuring data authenticity and accountability.

The combination of consumer-driven comparative effectiveness studies, AI-powered check-ins, and blockchain-based transparency creates a robust ecosystem for evaluating and promoting healthcare products based on the real-world evidence, thereby enhancing consumer trust and informed decision-making.

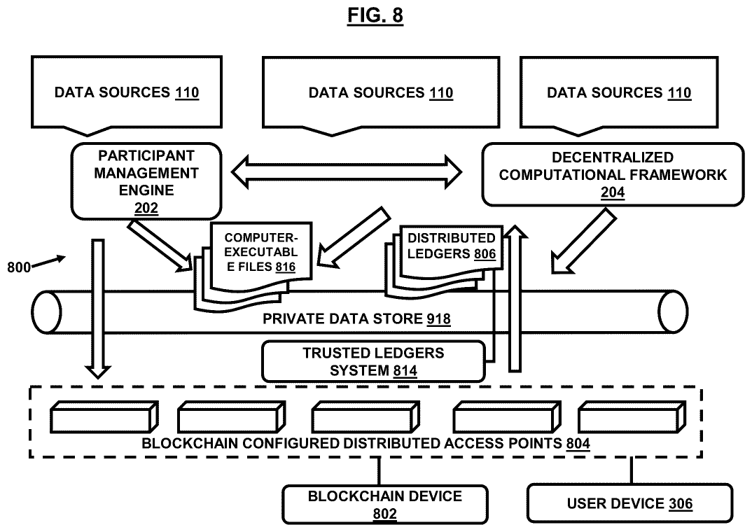

FIG. 8, with reference to FIGS. 1 through 7, illustrates an exemplary blockchain-configured ecosystem architecture 800 containing one or more components of the system 102 including such as the participant management engine 202 and the decentralized computational framework 204, and additional components to ensure the integrity of transactions and digital data (including information blocks and precision blocks) shared/processed during data transfer or storage, as previously discussed in this document without limitations. The blockchain-configured ecosystem architecture 800 provides a crowdsourced integrity network for storing data accessed, extracted, or transformed, for sharing or storing across a computer network. This approach contrasts with information stored locally by various participants or databases, which may be vulnerable to tampering.

The ecosystem architecture 800 may be blockchain-configured to incorporate various blockchain devices. For instance, the participant management engine 202 and the decentralized computational framework 204 may interact with a blockchain device 802 through a plurality of blockchain-configured distributed access points 804. A network facilitating interaction among all components may be a blockchain integrity network, fostering trust among various participants, entities, systems, and components thereof, and their associated computing terminals or devices, even if the devices/terminals or machines do not have direct knowledge of one another. The blockchain network enables secure connections, transactions, recording, and sharing of data, information blocks, precision blocks, and tokens, including service and authorization tokens, through a trusted system. The blockchain maintains a record of transactions, data exchanges, and updates from various terminals/devices on distributed ledgers 806, which provides a tamper-resistant history to build trust among terminals/devices (such as those associated with various participants or nodes) enabling peer-to-peer or peer-to-client interactions through a distributed digital ledger technology system. The ecosystem architecture 800 includes a distributed trusted ledger system 814 containing the distributed blockchain ledgers 806, which are associated with a plurality of computing terminals and devices. Each ledger may store a copy of computer-executable files 816, which contain data inputs related to the participants and the experimental study such as the trial protocol and the like, trial outcomes, continuously tracked and recorded real world data and evidence from the participants, and other information relevant to the experimental study. This distributed ledger system 814 allows for rule-based contracts that execute when specified conditions are met, enabling automated adjustments to the trial protocol and the interventions etc based on real-time data ingestion and updates.

The various computing terminals or devices in the network serve as distributed peer-to-peer nodes and connections. The participant management engine 202 and the decentralized computational framework 204 and other components thereof of the system 102 may be configured to process context inputs and information blocks through the blockchain network based on defined rules. Each terminal/device/node in the ecosystem architecture 800 may receive a copy of the blockchain, which may be automatically downloaded upon joining the blockchain integrity network, thereby decentralizing the network. Every permissioned node or device in the network may act as an administrator of the blockchain, participating voluntarily in the decentralized network.

The blockchain mitigates risks associated with centralized data storage by distributing data across the network, which may include the computer-executable files 816 containing information blocks pertaining to the participants and other entities of the experimental study etc, context inputs, context patterns, real world data and evidence, and various tokens/codes, including transaction codes. Security on the blockchain is achieved using encryption technology and validation mechanisms, which ensure data integrity. Public and private keys facilitate secure access, where a public key defines a user's address on the blockchain, and a private key enables access to digital assets within the network.

In an embodiment, the distributed ledgers 806 enable the coding of smart contracts (using smart contract systems) that execute upon the satisfaction of predefined conditions. These smart contracts may protect various data pieces associated with patient care and mitigate risks of unauthorized file copying and redistribution, ensuring patient privacy rights are maintained.

The blockchain-configured ecosystem architecture 800 may provide a private view for various devices and entities operating in the network through the private data store 818. This allows each permissioned device, such as the participant management engine 202 and the decentralized computational framework 204 and systems associated with the participants and administrators, to privately access the computer-executable files 816 associated with specific tasks, user inputs, and care directives based on permissions granted according to each entity's identity and roles defined by the system. The participant management engine 202 and the decentralized computational framework 204 and the systems (such as the system 102) associated with the participants and other entities may access these files through the private data store 818 via the distributed blockchain-configured access points 804, where each access point supports multi-party access based on defined rights.

The private data store 818 offers virtual storage to facilitate secure interaction, information exchange, review, and presentation of the computer-executable files 816. For example, the private data store 818 may allow the virtual presentation of only limited portions of executable files to specific entities, respecting permissions for access and review. The private data store 818 may be configured to auto-hash review interactions at defined intervals, ensuring that the computer-executable files 816 remain secure and private per access rights authorized to each node, while maintaining synchronization with permissioned access.

In an embodiment, the blockchain-configured digital ecosystem architecture 800 may incorporate a federated blockchain consisting of several entities/participants (including healthcare providers, administrators, and patients) and their associated devices. These components jointly interact to process data transfers through a trusted, secure, and distributed network of blockchain-configured access points 804.

In an embodiment, the blockchain device 802 may include the distributed trusted ledger system 814 containing the distributed blockchain ledgers 806 associated with the computing terminals. Each ledger stores a copy of computer-executable files that include data about the participants, the treatment protocol and other information about the participants and the experimental study. This blockchain enabled information provides a verifiable record of interactions within the system 102, supporting the system's objectives by ensuring data integrity and transparency through the blockchain.

In an example, the system 102 may be configured as an autonomous direct-to-consumer recruitment system 102 for the participants for the digitally executed experimental studies. The system 102 may include the server system 104 including the processor 106 and the memory circuit 108 storing instructions that, when executed, cause the processor 106 to receive, from a user via a user interface, the participant data in a computer-executable format. The participant data includes the participant-specific characteristics such as demographic information, medical history, and configuration details of the patient associated device 412 generating real world data. The processor 106 is configured to synchronize with the associated device 412 to automatically gather the real-world data from the user device, store the participant data and continuously update the real-world data of the user in the participant database. The processor 106 is configured to execute the machine learning algorithms 406 to analyze the participant data and continuously assess eligibility against the trial protocols associated with the experimental studies. The processor 106 matches the user to eligible studies when the stored data satisfies at least one of the trial protocols.

The processor 106 may be further configured to either receive or generate the plurality of the trial protocols defining the study-specific recruitment criteria, intervention details, outcome measures, and the inclusion and exclusion parameters for the digitally executed experimental studies.

The processor 106 may be further configured to generate and transmit the automated digital invitation to the matched user, for the enrolment into the matched experimental study upon the affirmative response.

The processor 106 is further configured to automatically onboard the user for the matched experimental study. The processor 106 is further configured to obtain the one or more tamper-proof informed consent records from the user using a blockchain-enabled data assurance device.

The processor 106 is further configured to initiate a real-time synchronization and update of the associated device to collect and transmit the real-world data including one or more of computer executable physiological data, computer executable nutrition data, and the computer executable lifestyle data of the user for use in the matched experimental study.

The user device may be the wearable device 318 generating the real world data.

The user device may be the mobile application 316 generating the real world data.

In an embodiment, the matched experimental study is a first study. The processor 106 is further configured to enroll the user in a second study of the experimental studies simultaneously such that the user participates in more than one experimental studies depending on matching based on the trial protocols associated with the experimental studies.

In some embodiments, the system 102 is configured as the autonomous direct-to-consumer recruitment system for the user for the digitally executed experimental studies. The system 102 includes the server system 104 including the processor 106 and the memory storing instructions that, when executed, cause the processor 106 to receive, from a user via a user interface, the participant data in a computer-executable format. The registration data includes the participant-specific characteristics comprising the demographic information, medical history, associated device configuration, and the real-time real-world data elements. The processor 106 is further configured to store the participant data in a participant database, receive the computer-executable trial protocols defining the study-specific participant recruitment criteria, intervention details, outcome measures, and the inclusion and exclusion parameters. The computer executable trial protocols are associated with the digitally executed experimental studies. The processor 106 is configured to execute the machine learning algorithms 406 configured to analyze and classify the participant data against the participant recruitment criteria associated with the digitally executed experimental studies, dynamically match the user to the trial protocols, generate and transmit, via the recruitment integration subsystem 306, the automated digital invitation to the user for the matched experimental study from the digitally executed experimental studies. The processor 106 is configured to receive and process a participant response to the automated digital invitation from the user for the matched experimental study, wherein an affirmative response triggers automatic onboarding for the matched experimental study.

The processor 106 is configured to dynamically match the user to the trial protocols associated with the experimental studies based on the probabilistic scoring models that evaluate compatibility of the user with the trial protocols.

The processor 106 is configured to generate and transmit the automated digital invitation to the user through the digital channels 314, including such as the mobile application 316, the wearable device 318 interface, the social networking server, and the patient portal, and the like without limitations.

In embodiments, the automatic onboarding may include obtaining one or more tamper-proof informed consent records from the user using a blockchain-enabled data assurance device, and initiating a real-time synchronization of the associated device, with the server system 104 to collect and transmit the real world data including the physiological data of the user for use in the matched experimental study.

The system 102 is further configured to continuously update the participant database with the newly ingested data from the associated device of the user, enabling reusability of participant profiles for subsequent studies and autonomously adapt the recruitment by updating the machine learning algorithms 406 based on historical participant engagement and recruitment outcomes.

The disclosed system integrates the real-world data (RWD) and the real-world evidence (RWE) directly into a clinical trial management workflow. This integration may enable continuous, real-time data ingestion from the plurality of data sources 110, including but not limited to the electronic health records (EHRs), the wearable devices 318, and the patient-reported outcomes. The system 102 may be configured to process this data dynamically throughout the lifecycle of the trial, ensuring timely and the actionable insights.

The system 102 may further incorporate various artificial intelligence (AI) and machine learning algorithms 406 including such as those discussed in this document without limitations to automatically clean, standardize, and analyze the ingested data in real-time. This automated processing may significantly reduce a manual data entry burden typically borne by principal investigators (PIs) and trial sponsors, who are otherwise required to compile and review disparate datasets post-trial. In contrast to conventional clinical trial management systems (CTMS) that lack native support for the RWD/RWE integration and rely on external systems or manual intervention for a post-trial analysis, the disclosed system may provide built-in, real-time RWD/RWE capabilities. This functionality may improve operational efficiency by reducing workload of PIs and sponsors, while also improving relevance and immediacy of data used to inform trial processes and outcomes.

In various embodiments, the system 102 may empower consumers and non-traditional service providers to define and execute comparative effectiveness studies and A/B tests, including such as both traditional and non-traditional interventions, such as dietary regimens and fitness protocols without limitations. The system 102 may allow non-drug and non-traditional treatments to be conducted as scientific studies similar to how medical device and drug studies are conducted. The system may be configured for real-time protocol adaptation powered by various machine learning algorithms such as those discussed above in the document, seamless integration of the diverse data sources which may include clinical and non-clinical domains and the like. The system 102 may facilitate automated regulatory compliance across multiple jurisdictions, enabling non-clinical professionals to design and conduct scientifically robust studies without requiring specialized expertise in clinical trial methodologies. The system may allow democratizing access to scientific-grade experimental studies, thereby allowing users to generate credible real-world evidence in support of various healthcare interventions.

The system 102 may empower consumers, including patients or the participants, to independently define and initiate comparative effectiveness studies tailored to their specific healthcare needs. The participants may be allowed to set criteria for the A/B testing of healthcare products or services, such as selecting intervention types, outcome measures, and participant characteristics through an intuitive interface. The participants may configure the experimental studies and track progress without requiring expertise in clinical study design. The system 102 may integrate the real-world data from such as the wearable devices, mobile applications, and self-reported metrics to evaluate effectiveness of the interventions. The system may include various advanced analytical tools and dashboards to provide the participants with actionable insights, allowing them to make informed decisions based on scientifically supported comparisons of their chosen products or services. This consumer-driven approach may help democratize access to evidence-based evaluations and advance personalized healthcare innovation.

In various embodiments, the experimental studies may be conducted by clinicians or trial investigators or patients or the participants who may not be clinicians or investigators by themselves. Users of the system 102 may include a wide array of non-traditional healthcare providers who contribute to holistic health and wellness. These may include, but are not limited to, wellness coaches, nutritionists, dietitians, fitness trainers, yoga instructors, mental health counselors, alternative medicine practitioners, physiotherapists, lifestyle consultants, naturopaths, acupuncturats, aromatherapists, health coaches, chiropractors, occupational therapists, meditation guides, and personal trainers. The system 102 may be designed to support these non-clinical professionals, empowering them to conduct scientifically robust evaluations of the interventions such as dietary plans, fitness regimens, mindfulness programs, and other consumer-focused wellness services. This inclusive definition of the users of the system 102 may help the growing adoption of integrative health approaches and highlight the system's ability to facilitate scientifically validated effectiveness studies for the interventions traditionally outside the purview of conventional medicine.

The integration of the system 102 with the wearable devices and other data sources generating the data improves its ability to conduct the comparative studies across a wide range of environments, including those outside traditional healthcare settings. The system 102, through continuously collecting the real-time physiological data or other data from the wearable devices and the mobile applications such as fitness trackers, smartwatches, and health monitors enables assessment of the interventions, such as fitness routines or dietary changes without limitations directly in the participants' everyday environments. This integration not only supports more accurate, real-world evaluations of these interventions but also broadens the scope of the comparative studies, thereby allowing non-clinical professionals and consumers to conduct scientifically rigorous research in settings outside of conventional healthcare institutions.

The ability of the system 102 to adapt the trial protocols in real-time based on the interim results provides important benefits for the users including the participants. The system 102 may use the machine learning algorithms to continuously analyze the incoming real-world data, such as participant health metrics and engagement, to dynamically adjust the interventions, eligibility criteria, recruitment strategies, and sample allocations and the like. The system 102 may be configured to optimize the trial protocols on the fly, ensuring that resources are efficiently allocated, and the participants are engaged with the most effective interventions. The system 102 may reduce trial duration and improve outcomes by responding more quickly to emerging patterns, compared to static, traditional trial methods that rely on pre-determined protocols and longer timelines for adjustments.

The system 102 may improve collaboration among multiple stakeholders, including such as patients, traditional clinicians, non-traditional providers (such as wellness coaches, nutritionists, and fitness experts), and trial sponsors. The system 102 may facilitate communication and data sharing and empower each stakeholder to contribute their expertise and insights to the trial process. For example, patients can engage with their healthcare providers, whether traditional or non-traditional, and share real-time data, while clinicians can monitor progress and make informed decisions. Trial sponsors can benefit from access to diverse participant pools and the ability to adjust trial parameters based on real-world data. This collaborative arrangement may encourage co-creation of trials, balancing the medical rigor required for scientific studies with the innovation and flexibility driven by consumer needs. This may allow development of personalized, effective interventions that reflect both clinical expertise and consumer-driven preferences, ultimately improving relevance and outcomes of the experimental studies.

The system 102 may allow incentivizing participation by offering micropayments, cryptocurrency, or reward points to the participants for adhering to the trial protocols. This may promote greater engagement and compliance, aligning with current consumer trends that favor flexible and rewarding participation models. The system may support providing tangible rewards, which can improve participant motivation.

The system 102 may be configured to address the growing concern of misinformation in healthcare by ensuring the integrity and scientific rigor of the experimental studies conducted utilizing the system 102. The system mitigates the risk of unverified healthcare claims being promoted, especially those derived from misleading or poorly designed A/B tests or experimental studies. The system 102 may achieve this by utilizing the real-world data and scientifically validated trial protocols, which are enforced through adaptive trial designs and AI-driven compliance mechanisms facilitated by the system 102, thereby ensuring that only scientifically sound studies are conducted.

In particular, the system 102 may leverage blockchain technology as discussed above to create an immutable record of data provenance and participant consent, which prevents tampering or manipulation of the outcomes. This blockchain integration ensures that all data is transparent and traceable, further protecting against dissemination of falsified or misleading claims. The system 102 may help non-scientists to conduct scientifically valid experimental studies, empowering them to design and run trials that adhere to established standards of scientific integrity.

Through the continuous real-time validation of data and the use of the adaptive trial protocols, the system 102 ensures that only evidence-based outcomes are generated, safeguarding the credibility of healthcare claims. The system 102 allows for the prevention of misinformation by ensuring that only accurate, validated, and scientifically rigorous comparative effectiveness studies are performed and reported. This allows the system 102 to actively combat proliferation of unverified healthcare claims, enhancing the transparency and trustworthiness of the data generated.

The system 102 provides specific technical improvements to overcome challenges in conducting digital experimental studies through its novel technical architecture and machine learning capabilities. The processor 106 is configured with specialized neural network models and reinforcement learning models 302 that enable dynamic protocol adaptation in real-time, which is a capability not possible with traditional manual trial management approaches. For example, the neural networks implemented within the machine learning algorithms 406 continuously analyze incoming physiological data streams from wearable devices 318 and patient-reported outcomes to automatically detect patterns and anomalies that may require protocol adjustments. The reinforcement learning models 302 are specifically trained on historical trial data to learn optimal adaptation strategies, considering multiple competing objectives like statistical power, patient safety, and resource utilization. This automated, AI-driven approach represents a concrete technological advance over existing systems that require manual monitoring and cannot dynamically optimize protocols.

The computer architecture provided by the system 102 also provides novel solutions for secure, privacy-preserving data integration across distributed environments. The decentralized computational framework 204 implements homomorphic encryption and differential privacy techniques within the data ingestion subsystem 402 to enable computations on encrypted data, allowing sensitive patient information to be processed without exposure. The blockchain-enabled ecosystem architecture 800 creates tamper-proof audit trails of all protocol changes, consent records, and outcomes using smart contracts implemented through distributed blockchain ledgers 806 that automatically enforce regulatory compliance rules. The interoperability framework 214 uses standardized FHIR specifications and API orchestration layer 702 to seamlessly combine data from electronic health record (EHR) systems 704, laboratory information management systems (LIMS) 708, and other sources while maintaining data lineage and provenance. These architectural components work together to create a technological platform that makes rigorous experimental studies accessible to non-traditional providers while ensuring security, compliance and scientific validity through technical means rather than manual processes.

The various components described herein and/or illustrated in the figures may be embodied as hardware-enabled modules and may be a plurality of overlapping or independent electronic circuits, devices, and discrete elements packaged onto a circuit board to provide data and signal processing functionality within a computer. An example might be a comparator, inverter, or flip-flop, which could include a plurality of transistors and other supporting devices and circuit elements. The modules that include electronic circuits process computer logic instructions capable of providing digital and/or analog signals for performing various functions as described herein. The various functions can further be embodied and physically saved as any of data structures, data paths, data objects, data object models, object files, database components. For example, the data objects could include a digital packet of structured data. Example data structures may include any of an array, tuple, map, union, variant, set, graph, tree, node, and an object, which may be stored and retrieved by computer memory and may be managed by processors, compilers, and other computer hardware components. The data paths can be part of a computer CPU that performs operations and calculations as instructed by the computer logic instructions. The data paths could include digital electronic circuits, multipliers, registers, and buses capable of performing data processing operations and arithmetic operations (e.g., Add, Subtract, etc.), bitwise logical operations (AND, OR, XOR, etc.), bit shift operations (e.g., arithmetic, logical, rotate, etc.), complex operations (e.g., using single clock calculations, sequential calculations, iterative calculations, etc.). The data objects may be physical locations in computer memory and can be a variable, a data structure, or a function. Some examples of the modules include relational databases (e.g., such as Oracle® relational databases), and the data objects can be a table or column, for example. Other examples include specialized objects, distributed objects, object-oriented programming objects, and semantic web objects. The data object models can be an application programming interface for creating HyperText Markup Language (HTML) and Extensible Markup Language (XML) electronic documents. The models can be any of a tree, graph, container, list, map, queue, set, stack, and variations thereof, according to some examples. The data object files can be created by compilers and assemblers and contain generated binary code and data for a source file. The database components can include any of tables, indexes, views, stored procedures, and triggers.

In an example, the embodiments herein can provide a computer program product configured to include a pre-configured set of instructions, which when performed, can result in actions as stated in conjunction with various figures herein. In an example, the pre-configured set of instructions can be stored on a tangible non-transitory computer readable medium. In an example, the tangible non-transitory computer readable medium can be configured to include the set of instructions, which when performed by a device, can cause the device to perform acts similar to the ones described here.

The embodiments herein may also include tangible and/or non-transitory computer-readable storage media for carrying or having computer-executable instructions or data structures stored thereon. Such non-transitory computer readable storage media can be any available media that can be accessed by a general purpose or special purpose computer, including the functional design of any special purpose processor as discussed above.

By way of example, and not limitation, such non-transitory computer-readable media can include RAM, ROM, EEPROM, CD-ROM or other optical disk storage, magnetic disk storage or other magnetic storage devices, or any other medium which can be used to carry or store desired program code means in the form of computer-executable instructions, data structures, or processor chip design. When information is transferred or provided over a network or another communications connection (either hardwired, wireless, or combination thereof) to a computer, the computer properly views the connection as a computer-readable medium. Thus, any such connection is properly termed a computer-readable medium. Combinations of the above should also be included within the scope of the computer-readable media.

Computer-executable instructions include, for example, instructions and data which cause a special purpose computer or special purpose processing device to perform a certain function or group of functions. Computer-executable instructions also include program modules that are executed by computers in stand-alone or network environments. Generally, program modules include routines, programs, components, data structures, objects, and the functions inherent in the design of special-purpose processors, etc. that perform particular tasks or implement particular abstract data types. Computer-executable instructions, associated data structures, and program modules represent examples of the program code means for executing steps of the methods disclosed herein. The particular sequence of such executable instructions or associated data structures represents examples of corresponding acts for implementing the functions described in such steps.

The techniques provided by the embodiments herein may be implemented on an integrated circuit chip (not shown). The chip design is created in a graphical computer programming language, and stored in a computer storage medium (such as a disk, tape, physical hard drive, or virtual hard drive such as in a storage access network. If the designer does not fabricate chips or the photolithographic masks used to fabricate chips, the designer transmits the resulting design by physical means (e.g., by providing a copy of the storage medium storing the design) or electronically (e.g., through the Internet) to such entities, directly or indirectly. The stored design is then converted into the appropriate format (e.g., GDSII) for the fabrication of photolithographic masks, which typically include multiple copies of the chip design in question that are to be formed on a wafer. The photolithographic masks are utilized to define areas of the wafer (and/or the layers thereon) to be etched or otherwise processed.

The resulting integrated circuit chips can be distributed by the fabricator in raw wafer form (that is, as a single wafer that has multiple unpackaged chips), as a bare die, or in a packaged form. In the latter case the chip is mounted in a single chip package (such as a plastic carrier, with leads that are affixed to a motherboard or other higher level carrier) or in a multichip package (such as a ceramic carrier that has either or both surface interconnections or buried interconnections). In any case the chip is then integrated with other chips, discrete circuit elements, and/or other signal processing devices as part of either (a) an intermediate product, such as a motherboard, or (b) an end product. The end product can be any product that includes integrated circuit chips, ranging from toys and other low-end applications to advanced computer products having a display, a keyboard or other input device, and a central processor.

Furthermore, the embodiments herein can take the form of a computer program product accessible from a computer-usable or computer-readable medium providing program code for use by or in connection with a computer or any instruction execution system. For the purposes of this description, a computer-usable or computer readable medium can be any apparatus that can comprise, store, communicate, propagate, or transport the program for use by or in connection with the instruction execution system, apparatus, or device.

The medium can be an electronic, magnetic, optical, electromagnetic, infrared, or semiconductor system (or apparatus or device) or a propagation medium. Examples of a computer-readable medium include a semiconductor or solid-state memory, magnetic tape, a removable computer diskette, a random access memory (RAM), a read-only memory (ROM), a rigid magnetic disk and an optical disk. Current examples of optical disks include compact disk-read only memory (CD-ROM), compact disk-read/write (CD-R/W) and DVD.

A data processing system suitable for storing and/or executing program code will include at least one processor coupled directly or indirectly to memory elements through a system bus. The memory elements can include local memory employed during actual execution of the program code, bulk storage, and cache memories which provide temporary storage of at least some program code in order to reduce the number of times code must be retrieved from bulk storage during execution.

Input/output (I/O) devices (including but not limited to keyboards, displays, pointing devices, etc.) can be coupled to the system either directly or through intervening I/O controllers. Network adapters may also be coupled to the system to enable the data processing system to become coupled to other data processing systems or remote printers or storage devices through intervening private or public networks. Modems, cable modem and Ethernet cards are just a few of the currently available types of network adapters.

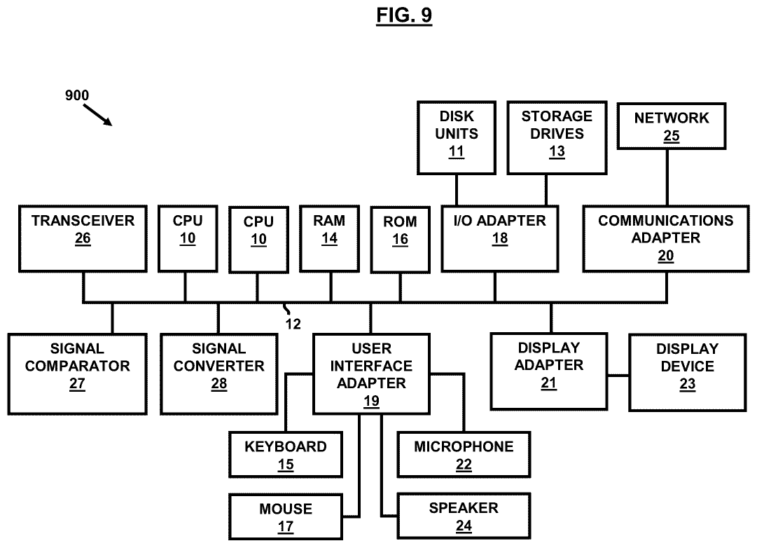

A representative hardware environment for practicing the embodiments herein is depicted in FIG. 9, with reference to FIGS. 1 through 8. This schematic drawing illustrates a hardware configuration of an information handling/computer system 900 in accordance with the embodiments herein.

The system 900 comprises at least one processor or central processing unit (CPU) 10. The CPUs 10 are interconnected via system bus 12 to various devices such as a random access memory (RAM) 14, read-only memory (ROM) 16, and an input/output (I/O) adapter 18. The I/O adapter 18 can connect to peripheral devices, such as disk units 11 and tape drives 13, or other program storage devices that are readable by the system. The system 900 can read the inventive instructions on the program storage devices and follow these instructions to execute the methodology of the embodiments herein. The system 900 further includes a user interface adapter 19 that connects a keyboard 15, mouse 17, speaker 24, microphone 22, and/or other user interface devices such as a touch screen device (not shown) to the bus 12 to gather user input. Additionally, a communication adapter 20 connects the bus 12 to a data processing network, and a display adapter 21 connects the bus 12 to a display device 23 which may be embodied as an output device such as a monitor, printer, or transmitter, for example. Further, a transceiver 26, a signal comparator 27, and a signal converter 28 may be connected with the bus 12 for processing, transmission, receipt, comparison, and conversion of electric or electronic signals.

The system 102 may be implemented as a web-based application accessible through standard internet browsers, with the server system 104 deploying the application through secure HTTPS protocols. The user interface, which may be presented on the display device 23, may include the responsive dashboard 610 that dynamically adapts its layout and visualization components based on the user's role and device characteristics. For investigators, the dashboard 610 presents an interactive protocol builder with drag-and-drop functionality for defining study parameters, while participants see a simplified interface optimized for data entry and progress tracking. The web application may leverage WebSocket connections for real-time data streaming from wearable devices 318 and implement progressive web app (PWA) capabilities to enable offline functionality. The patient portal 322 may employ responsive design principles to automatically adjust its presentation across desktop, tablet, and mobile form factors while maintaining consistent access to study information and data collection tools. Custom web components may be utilized to create reusable interface elements specific to experimental study workflows, such as protocol visualization widgets, participant enrollment forms, and real-time monitoring displays. This web-centric architecture enables broad accessibility while maintaining the security and functionality required for rigorous experimental studies.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Terminology used herein should not be construed as being “means-plus-function” language unless the term “means” is expressly used in association therewith. Those skilled in the art will recognize that the embodiments herein can be practiced with modification within the spirit and scope of the appended claims.

What is claimed is:

1. An adaptive software-as-a-service (SaaS)-based system for autonomously managing an experimental study using real-world data, the system comprising:
a processor to:
generate or receive a trial protocol defining participant criteria, one or more interventions, one or more outcomes, and respective durations;
facilitate recruitment of one or more participants via one or more digital channels based on assessment of eligibility against the participant criteria; and
continuously analyze the real-world data associated with the one or more participants for one or more of adaptive trial adjustments, anomaly detection, and outcome generation.

2. The system of claim 1, wherein the processor is to refine the protocol using machine learning algorithms based on an interim result of the experimental study.

3. The system of claim 1, wherein the recruitment is performed dynamically by assessing the eligibility using real-time physiological data and probabilistic models.

4. The system of claim 1, wherein the one or more digital channels comprises one of a mobile application, a wearable device, and a social networking server.

5. The system of claim 1, wherein the processor is to enroll the one or more participants based on the participant criteria.

6. The system of claim 1, wherein the experimental study is a digitally conducted A/B test, the one or more interventions comprising at least a first intervention and a second intervention, wherein both the first intervention and the second intervention are delivered to at least one of the one or more participants such that at least one same participant receive both the first intervention and the second intervention.

7. The system of claim 6, wherein the one or more outcomes comprises a first outcome corresponding to the first intervention and a second outcome corresponding to the second intervention, wherein the processor is to analyze the first outcome and the second outcome to generate a computer executable comparative report.

8. The system of claim 1, wherein the processor is to:
implement a controlled comparative testing mechanism by allocating the one or more participants into at least two groups, wherein each group receives distinct interventions from the one or more interventions defined in the trial protocols; and
collect and analyze the real-world data from participants in each group, including the outcomes and the real-world time comprising physiological parameters, to evaluate comparative effectiveness of the distinct interventions.

9. The system of claim 8, wherein the processor is to generate an adaptive trial protocol based on an analysis from the at least two groups to optimize an intervention efficacy.

10. An adaptive software-as-a-service (SaaS)-based digital system for conducting an autonomous experimental study using real-world data, the system comprising:
a processor to execute machine-readable instructions stored on a non-transitory computer-readable medium, wherein the processor is to:
one of either receive, from a user interface, a trial protocol specifying one or more of participant recruitment criteria, intervention types, outcome measures, and trial duration, or autonomously generate the trial protocol by applying one or more machine learning algorithms to analyze historical study data and multi-dimensional input criteria;
facilitate recruitment of one or more participants by integrating with one or more digital channels, including one or more of a mobile application, a wearable device, and a social networking server;
dynamically assess participant eligibility by processing at least one of real-time physiological measurements and participant-reported inputs;
automatically enroll the one or more participants into the experimental study when their data satisfies the participant recruitment criteria specified in the trial protocol; and
execute computational models for analyzing the real-world data during the experimental study to generate one or more trial outcomes.

11. The system of claim 10, wherein the processor is to apply predictive models to monitor safety and efficacy of one or more interventions, detect one or more anomalies, and predict one or more adverse events.

12. A method for autonomously managing an experimental study using real-world data through an adaptive software-as-a-service (SaaS)-based system, the method comprising:
generating or receiving a trial protocol, wherein the trial protocol specifies participant criteria, one or more interventions, an outcome, and a duration of the experimental study;
assessing eligibility of the one or more participants against the participant criteria specified in the trial protocol;
facilitating recruitment of the one or more participants through one or more digital channels; and
continuously analyzing the real-world data associated with the one or more participants during the experimental study to perform one or more of adaptive trial adjustments, detecting one or more anomalies, and recording one or more outcomes.

13. The method of claim 12, wherein the analysis is conducted using one or more computational models configured for real-time data processing and decision-making.

14. The method of claim 12, comprising adjusting the trial protocol based on at least one of the one or more anomalies and the one or more adverse events.

15. The method of claim 12, comprising presenting real-time insights and the one or more trial outcomes through an interactive dashboard.

16. The method of claim 12, wherein the recruitment of the one or more participants is performed dynamically by assessing the eligibility using at least one of real-time physiological data and probabilistic models.

17. The method of claim 12, wherein the one or more digital channels comprises one of a mobile application, a wearable device, and a social networking server.

18. The method of claim 17, comprising:
implementing a controlled comparative testing mechanism by allocating the one or more participants into at least two groups, wherein each group receives distinct interventions from the one or more interventions defined in the trial protocols; and
collecting and analyzing the real-world data from participants in each group, including the outcomes and the real-world data comprising physiological parameters, to evaluate comparative effectiveness of the distinct interventions.

19. The system of claim 18, comprising generating an adaptive trial protocol based on an analysis from the at least two groups to optimize an intervention efficacy.

20. The method of claim 19, comprising automatically allocating the one or more participants to the experimental study matching their eligibility, wherein the allocation includes multi-group intervention comparisons.
